(19) United States
(12) Patent Application Publication
(10) Pub. No.: US 2024/0390260 A1
(43) Pub. Date: Nov. 28, 2024

(54) METHODS FOR ENHANCING SKIN RESISTANCE, IMPROVING SKIN SMOOTHNESS, OR IMPROVING SKIN BRIGHTNESS BY USING AMPELOPSIS GROSSEDENTATA LEAF EXTRACT

(71) Applicant: TCI CO., LTD., Taipei (TW)

(72) Inventors: YUNG-HSIANG LIN, Taipei (TW); HSIAO-NAI LIN, Taipei (TW)

(21) Appl. No.: 18/426,360

(22) Filed: Jan. 30, 2024

Related U.S. Application Data
(60) Provisional application No. 63/503,503, filed on May 22, 2023.

(30) Foreign Application Priority Data
Dec. 22, 2023 (TW) 112150455

(51) Int. Cl. A61K 8/9789; A61Q 17/04; A61Q 19/00; A61Q 19/02; A61Q 19/08

(52) U.S. Cl. CPC A61K 8/9789; A61Q 17/04; A61Q 19/007; A61Q 19/02; A61Q 19/08

(57) ABSTRACT

Methods for enhancing skin resistance, improving skin smoothness, or improving skin brightness are provided. The method includes administering to a subject in need thereof a composition containing Ampelopsis grossedentata leaf extract. The Ampelopsis grossedentata leaf extract is obtained by extracting leaves of Ampelopsis grossedentata with water at 85±5° C. for 50-70 min.

### CROSS-REFERENCES TO RELATED APPLICATIONS

This application claims the benefit of U.S. provisional application Ser. No. 63/503,503, filed on May 22, 2023 and claims the priority to patent application No. 112150455 filed in Taiwan, R.O.C. on Dec. 22, 2023. The entirety of the above-mentioned patent applications are hereby incorporated by references herein and made a part of the specification.

### REFERENCE OF ELECTRONIC SEQUENCE LISTING

The contents of the electronic sequence listing (P234551USI.xml; Size: 8,163 bytes; and Date of Creation: Jan. 23, 2024) is herein incorporated by reference in its entirety.

### BACKGROUND

### Technical Field

The present disclosure relates to a method for improving skin condition, and particularly relates to a method for improving skin condition by using Ampelopsis grossedentata leaf extract.

### Related Art

Since the rise of the concept of organic and/or natural diet, biotechnology companies and food industries have actively invested in the research and development of products related to natural plants. In order to provide a scientific proven basis for the benefits of plant-related products to human health, the analysis and efficacy evaluation of active ingredients in plants have become a key project in product development.

Ampelopsis grossedentata is a precious plant survived after the quaternary extinction event, and currently only survives in Zhangjiajie, Hunan Province. Ampelopsis grossedentata grows on extremely harsh mountain ridges at an altitude of 800-1,500 meters, and in special red sandstone soil, which makes it rich in antioxidants. Biotechnology companies and food industries actively perform research on the active ingredient analysis and efficacy evaluation of Ampelopsis grossedentata and develop related products accordingly.

### SUMMARY

In some embodiments, a use of an Ampelopsis grossedentata leaf extract in preparation of a composition for enhancing skin resistance is provided. The Ampelopsis grossedentata leaf extract is obtained by extracting leaves of Ampelopsis grossedentata with water at 85±5° C. for 50-70 min.

In some embodiments, a method for enhancing skin resistance is provided, including administering to a subject in need thereof a composition including an Ampelopsis grossedentata leaf extract. The Ampelopsis grossedentata leaf extract is obtained by extracting leaves of Ampelopsis grossedentata with water at 85±5° C. for 50-70 min.

In some embodiments, the Ampelopsis grossedentata leaf extract improves an antioxidant capability of skin of the subject.

In some embodiment, the Ampelopsis grossedentata leaf extract reduces DNA damage to skin cell of the subject.

In some embodiments, the Ampelopsis grossedentata leaf extract provides an anti-inflammation capability for skin of the subject.

In some embodiments, the Ampelopsis grossedentata leaf extract reduces nitric oxide level in skin cells of the subject.

In some embodiments, the Ampelopsis grossedentata leaf extract provides an ultraviolet ray resistance capability for skin of the subject.

In some embodiments, the Ampelopsis grossedentata leaf extract reduces damage to skin of the subject caused by ultraviolet ray.

In some embodiments, the Ampelopsis grossedentata leaf extract improves viability of skin cells of the subject under ultraviolet ray.

In some embodiments, the Ampelopsis grossedentata leaf extract reduces mortality of skin cells of the subject under ultraviolet ray.

In some embodiments, the Ampelopsis grossedentata leaf extract inhibits skin laxity of the subject caused by ultraviolet ray.

In some embodiments, the Ampelopsis grossedentata leaf extract reduces level of Matrix metalloproteinase 9 (MMP9) of skin cells of the subject under ultraviolet ray.

In some embodiments, a use of an Ampelopsis grossedentata leaf extract in preparation of a composition for improving skin smoothness is provided. The Ampelopsis grossedentata leaf extract is obtained by extracting leaves of Ampelopsis grossedentata with water at 85±5° C. for 50-70 min.

In some embodiments, a method for improving skin smoothness is provided, including administering to a subject in need thereof a composition including an Ampelopsis grossedentata leaf extract. The Ampelopsis grossedentata leaf extract is obtained by extracting leaves of Ampelopsis grossedentata with water at 85±5° C. for 50-70 min.

In some embodiments, the Ampelopsis grossedentata leaf extract reduces skin roughness of the subject.

In some embodiments, the Ampelopsis grossedentata leaf extract increases elastin expression of skin cells of the subject.

In some embodiments, the Ampelopsis grossedentata leaf extract reduces skin wrinkles of the subject.

In some embodiments, the Ampelopsis grossedentata leaf extract improves skin texture of the subject.

In some embodiments, the Ampelopsis grossedentata leaf extract increases skin hydration of the subject.

In some embodiment, a use of an Ampelopsis grossedentata leaf extract in preparation of a composition for improving skin brightness is provided. The Ampelopsis grossedentata leaf extract is obtained by extracting leaves of Ampelopsis grossedentata with water at 85±5° C. for 50-70 min.

In some embodiments, a method for improving skin brightness is provided, including administering to a subject in need thereof a composition including an Ampelopsis grossedentata leaf extract. The Ampelopsis grossedentata leaf extract is obtained by extracting leaves of Ampelopsis grossedentata with water at 85±5° C. for 50-70 min.

In some embodiments, the Ampelopsis grossedentata leaf extract reduces melanin production in skin cells of the subject.

In some embodiments, the Ampelopsis grossedentata leaf extract reduces expression level of at least one gene of the subject. The at least one gene is at least one of a TYR gene, a TYRP1 gene, an MC1R gene, and an M1TF gene.

In some embodiments, the Ampelopsis grossedentata leaf extract reduces spots on skin of the subject.

In some embodiments, the Ampelopsis grossedentata leaf extract fades superficial spots on the skin of the subject.

In some embodiments, the Ampelopsis grossedentata leaf extract fades brown spots on the skin of the subject.

In conclusion, the Ampelopsis grossedentata leaf extract according to the embodiments of the present disclosure has a skin condition improving effect. In some embodiments, a use of the Ampelopsis grossedentata leaf extract according to the embodiments of the present disclosure in improving skin condition relates to a use of the Ampelopsis grossedentata leaf extract in preparation of a composition for improving skin condition, thereby providing a composition capable of realizing the skin condition improving effect on an individual when being administered to the individual. In some embodiments, a method for improving skin condition includes: administering to a subject in need thereof a composition including the Ampelopsis grossedentata leaf extract according to the embodiments of the present disclosure. That is, the composition has the skin condition improving function. That is, the composition can improve the skin condition of the individual after being administered to the individual. In some embodiments, the Ampelopsis grossedentata leaf extract or the prepared composition thereof also has one or more of the following functions: enhancing skin resistance, improving skin smoothness, and improving skin brightness. In some embodiments, methods for enhancing skin resistance, improving skin smoothness, and improving skin brightness include: administering to a subject in need thereof a composition including the Ampelopsis grossedentata leaf extract.

### BRIEF DESCRIPTION OF THE DRAWINGS

FIG. 1 is a bar chart showing the relative cell viability after treated by Ampelopsis grossedentata leaf extract in accordance with some embodiments of the present disclosure.

FIG. 2 is a bar chart showing the relative apoptosis rate after treated by Ampelopsis grossedentata leaf extract in accordance with some embodiments of the present disclosure.

FIG. 3 is a bar chart showing the relative MMP9 level after treated by Ampelopsis grossedentata leaf extract in accordance with some embodiments of the present disclosure.

FIG. 4 is a bar chart showing the relative Phospho-H2AX level after treated by Ampelopsis grossedentata leaf extract in accordance with some embodiments of the present disclosure.

FIG. 5 is a bar chart showing the relative elastin level after treated by Ampelopsis grossedentata leaf extract in accordance with some embodiments of the present disclosure.

FIG. 6 is a bar chart showing the relative gene expression level after treated by Ampelopsis grossedentata leaf extract in accordance with some embodiments of the present disclosure.

FIG. 7 is a bar chart showing the relative nitric oxide level after treated by Ampelopsis grossedentata leaf extract in accordance with some embodiments of the present disclosure.

FIG. 8 is a bar chart showing the relative skin spot level in human subjects at week 0 and week 2 after ingesting a composition including Ampelopsis grossedentata leaf extract in accordance with some embodiments of the present disclosure.

FIG. 9 is a bar chart showing the relative skin brown spot level in human subjects at week 0 and week 2 after ingesting a composition including Ampelopsis grossedentata leaf extract in accordance with some embodiments of the present disclosure.

FIG. 10 is a bar chart showing the relative skin wrinkle level in human subjects at week 0 and week 2 after ingesting a composition including Ampelopsis grossedentata leaf extract in accordance with some embodiments of the present disclosure.

FIG. 11 is a bar chart showing the relative skin texture level in human subjects at week 0 and week 2 after ingesting a composition including Ampelopsis grossedentata leaf extract in accordance with some embodiments of the present disclosure.

FIG. 12 is a bar chart showing the relative skin hydration in human subjects at week 0 and week 2 after ingesting a composition including Ampelopsis grossedentata leaf extract in accordance with some embodiments of the present disclosure.

### DETAILED DESCRIPTION

In some embodiments, an Ampelopsis grossedentata leaf extract is obtained by performing an extraction process on leaves of Ampelopsis grossedentata serving as a raw material. The extraction process mainly includes: extracting the leaves of Ampelopsis grossedentata with a solvent to dissolve effective ingredients in the leaves of the Ampelopsis grossedentata into the solvent.

In some embodiments, the extracted leaves of Ampelopsis grossedentata may be leaves that are intact and not physically pre-treated (the size of the leaves is not physically pre-treated), or may be decomposed into small-size forms such as fragments, particles or powder through physical pretreatment. The physical pretreatment may include at least one of coarse crushing, chopping, shearing, mashing and grinding. In some embodiments, the extracted leaves of Ampelopsis grossedentata are ground leaves.

In some embodiments, the extracted leaves may be just collected leaves, dried leaves and/or frozen leaves. For example, in the extraction process, the dried leaves are extracted with the solvent.

In some embodiments, the extraction process includes the step of extracting the leaves of Ampelopsis grossedentata with water at 70-90° C. for 50-70 min to obtain primary extract. For example, the leaves of Ampelopsis grossedentata may be soaked in water at 85±5° C. for 60 min to dissolve the effective ingredients in the leaves of Ampelopsis grossedentata into the water so as to obtain the primary extract.

In some embodiments, in the extraction process, the solvent is water, the raw material is the leaves of Ampelopsis grossedentata, and the weight ratio of the solvent to the raw material in primary mixing is (10-30):1. For example, the weight ratio of the water to the leaves of Ampelopsis grossedentata is 20:1.

In some embodiments, in the extraction process, the primary extract may be further filtered to remove solids such as the leaves of Ampelopsis grossedentata subjected to water extraction so as to obtain filtrate. For example, the primary extract may be filtered with a 400-mesh filter to remove the solids, and the filtered filtrate is collected.

In some embodiments, in the extraction process, the filtrate may be further concentrated to obtain a concentrated solution. In some embodiments, the filtrate may be concentrated under reduced pressure at 45-65° C. to obtain the concentrated solution. For example, the filtrate may be concentrated under reduced pressure at 60±5° C. In some embodiments, the duration for concentration may be determined by Degrees Brix of the concentrated solution, but it is not limited to this. Following the previous example, the Degrees Brix of the obtained concentrated solution is 6.5±0.5° Bx. That is, the filtrate may be concentrated under reduced pressure at 60±5° C. until the filtrate has a Bx of 6.5±0.5°, and the resulting filtrate concentrated under reduced pressure is the concentrated solution.

In some embodiments, in the extraction process, the primary extract may be first concentrated to form a concentrated solution with reduced volume. Then the concentrated solution is filtered to remove solids in the concentrated solution, and the filtered filtrate is collected.

In some embodiments, in the extraction process, the primary extract may also be concentrated without filtration or filtered without concentration.

It is to be understood that the primary extract, the filtrate, the concentrated solution or any combination thereof obtained in the extraction process may be used as the Ampelopsis grossedentata leaf extract according to actual needs.

In some embodiments, the Ampelopsis grossedentata leaf extract has a capability of enhancing skin resistance. Therefore, the Ampelopsis grossedentata leaf extract is suitable for preparing a composition for enhancing skin resistance.

In some embodiments, a method for enhancing skin resistance is provided, including administering to a subject in need thereof a composition including an Ampelopsis grossedentata leaf extract.

In some embodiments, the Ampelopsis grossedentata leaf extract has a capability of improving an antioxidant capability of skin. That is, the Ampelopsis grossedentata leaf extract administered to an individual can improve an antioxidant capability of skin of the individual. Therefore, the Ampelopsis grossedentata leaf extract is suitable for preparing a composition for improving an antioxidant capability of skin.

In some embodiments, the Ampelopsis grossedentata leaf extract has a capability of reducing damage to skin cell DNA. That is, the Ampelopsis grossedentata leaf extract administered to an individual can reduce damage to skin cell DNA of the individual. Therefore, the Ampelopsis grossedentata leaf extract is suitable for preparing a composition for reducing damage to skin cell DNA.

In some embodiments, the Ampelopsis grossedentata leaf extract has a capability of providing an anti-inflammation capability for skin. That is, the Ampelopsis grossedentata leaf extract administered to an individual can provide an anti-inflammation capability for skin of the individual. Therefore, the Ampelopsis grossedentata leaf extract is suitable for preparing a composition for providing an anti-inflammation capability for skin.

In some embodiments, the Ampelopsis grossedentata leaf extract has a capability of reducing nitric oxide level in skin cells. That is, the Ampelopsis grossedentata leaf extract administered to an individual can reduce nitric oxide level in skin cells of the individual. Therefore, the Ampelopsis grossedentata leaf extract is suitable for preparing a composition for reducing nitric oxide level in skin cells.

In some embodiments, the Ampelopsis grossedentata leaf extract has a capability of providing an ultraviolet ray resistance capability for skin. That is, the Ampelopsis grossedentata leaf extract administered to an individual can provide an ultraviolet ray resistance capability for skin of the individual. Therefore, the Ampelopsis grossedentata leaf extract is suitable for preparing a composition for providing an ultraviolet ray resistance capability for skin.

In some embodiments, the Ampelopsis grossedentata leaf extract has a capability of reducing damage to skin caused by ultraviolet ray. That is, the Ampelopsis grossedentata leaf extract administered to an individual can reduce damage to skin of the individual caused by ultraviolet ray. Therefore, the Ampelopsis grossedentata leaf extract is suitable for preparing a composition for reducing damage to skin caused by ultraviolet ray.

In some embodiments, the Ampelopsis grossedentata leaf extract has a capability of improving viability of skin cells under ultraviolet ray. That is, the Ampelopsis grossedentata leaf extract administered to an individual can improve viability of skin cells of the individual under ultraviolet ray. Therefore, the Ampelopsis grossedentata leaf extract is suitable for preparing a composition for improving viability of skin cells under ultraviolet ray.

In some embodiments, the Ampelopsis grossedentata leaf extract has a capability of reducing mortality of skin cells under ultraviolet ray. That is, the Ampelopsis grossedentata leaf extract administered to an individual can reduce mortality of skin cells of the individual under ultraviolet ray. Therefore, the Ampelopsis grossedentata leaf extract is suitable for preparing a composition for reducing mortality of skin cells under ultraviolet ray.

In some embodiments, the Ampelopsis grossedentata leaf extract has a capability of inhibiting skin laxity caused by ultraviolet ray. That is, the Ampelopsis grossedentata leaf extract administered to an individual can inhibit skin laxity of the individual caused by ultraviolet ray. Therefore, the Ampelopsis grossedentata leaf extract is suitable for preparing a composition for inhibiting skin laxity caused by ultraviolet ray.

In some embodiments, the Ampelopsis grossedentata leaf extract has a capability of reducing level of Matrix metalloproteinase 9 (MMP9) of skin cells under ultraviolet ray. That is, the Ampelopsis grossedentata leaf extract administered to an individual can reduce level of MMP9 of skin cells of the individual under ultraviolet ray. Therefore, the Ampelopsis grossedentata leaf extract is suitable for preparing a composition for reducing level of MMP9 of skin cells under ultraviolet ray.

In some embodiments, a method for improving an antioxidant capability of skin, reducing damage to skin cell DNA, providing an anti-inflammation capability for skin, reducing nitric oxide level in skin cells, providing an ultraviolet ray resistance capability for skin, reducing damage to skin caused by ultraviolet ray, improving viability of skin cells under ultraviolet ray, reducing mortality of skin cells under ultraviolet ray, inhibiting skin laxity caused by ultraviolet ray, reducing level of Matrix metalloproteinase 9 (MMP9) of skin cells under ultraviolet ray or any combination thereof includes: administering to a subject in need thereof a composition including an Ampelopsis grossedentata leaf extract.

In some embodiments, the Ampelopsis grossedentata leaf extract has a capability of improving skin smoothness. Therefore, the Ampelopsis grossedentata leaf extract is suitable for preparing a composition for improving skin smoothness.

In some embodiments, a method for improving skin smoothness is provided, including administering to a subject in need thereof a composition including an Ampelopsis grossedentata leaf extract.

In some embodiments, the Ampelopsis grossedentata leaf extract has a capability of reducing skin roughness. That is, the Ampelopsis grossedentata leaf extract administered to an individual can reduce skin roughness of the individual. Therefore, the Ampelopsis grossedentata leaf extract is suitable for preparing a composition for reducing skin roughness.

In some embodiments, the Ampelopsis grossedentata leaf extract has a capability of increasing elastin expression of skin cells. That is, the Ampelopsis grossedentata leaf extract administered to an individual can increase elastin expression of skin cells of the individual. Therefore, the Ampelopsis grossedentata leaf extract is suitable for preparing a composition for increasing elastin expression of skin cells.

In some embodiments, the Ampelopsis grossedentata leaf extract has a capability of reducing skin wrinkles. That is, the Ampelopsis grossedentata leaf extract administered to an individual can reduce skin wrinkles of the individual. Therefore, the Ampelopsis grossedentata leaf extract is suitable for preparing a composition for reducing skin wrinkles.

In some embodiments, the Ampelopsis grossedentata leaf extract has a capability of improving skin texture. That is, the Ampelopsis grossedentata leaf extract administered to an individual can improve skin texture of the individual. Therefore, the Ampelopsis grossedentata leaf extract is suitable for preparing a composition for improving skin texture.

In some embodiments, the Ampelopsis grossedentata leaf extract has a capability of increasing skin hydration. That is, the Ampelopsis grossedentata leaf extract administered to an individual can increase skin hydration of the individual. Therefore, the Ampelopsis grossedentata leaf extract is suitable for preparing a composition for increasing skin hydration.

In some embodiments, a method for reducing skin roughness, increasing elastin expression of skin cells, reducing skin wrinkles, improving skin texture, increasing skin hydration or any combination thereof includes: administering to a subject in need thereof a composition including an Ampelopsis grossedentata leaf extract.

In some embodiments, the Ampelopsis grossedentata leaf extract has a capability of improving skin brightness. Therefore, the Ampelopsis grossedentata leaf extract is suitable for preparing a composition for improving skin brightness.

In some embodiments, a method for improving skin brightness is provided, including administering to a subject in need thereof a composition including an Ampelopsis grossedentata leaf extract.

In some embodiments, the Ampelopsis grossedentata leaf extract has a capability of reducing melanin production in skin cells. That is, the Ampelopsis grossedentata leaf extract administered to an individual can reduce melanin production in the skin cells of the individual. Therefore, the Ampelopsis grossedentata leaf extract is suitable for preparing a composition for reducing melanin production in skin cells.

In some embodiments, the Ampelopsis grossedentata leaf extract has a capability of reducing expression level of at least one gene. That is, the Ampelopsis grossedentata leaf extract administered to an individual can reduce expression level of at least one gene of the individual. Therefore, the Ampelopsis grossedentata leaf extract is suitable for preparing a composition for reducing expression level of at least one gene. The at least one gene is at least one of a TYR gene, a TYRP1 gene, an MC1R gene and an M1TF gene.

In some embodiments, the Ampelopsis grossedentata leaf extract has a capability of reducing spots on skin. That is, the Ampelopsis grossedentata leaf extract administered to an individual can reduce spots on skin of the individual. Therefore, the Ampelopsis grossedentata leaf extract is suitable for preparing a composition for reducing spots on skin.

In some embodiments, the Ampelopsis grossedentata leaf extract has a capability of fading superficial spots on skin. That is, the Ampelopsis grossedentata leaf extract administered to an individual can fade superficial spots on skin of the individual. Therefore, the Ampelopsis grossedentata leaf extract is suitable for preparing a composition for fading superficial spots on skin.

In some embodiments, the Ampelopsis grossedentata leaf extract has a capability of fading brown spots on skin. That is, the Ampelopsis grossedentata leaf extract administered to an individual can fade brown spots on skin of the individual. Therefore, the Ampelopsis grossedentata leaf extract is suitable for preparing a composition for fading brown spots on skin.

In some embodiments, a method for reducing melanin production in skin cells, reducing expression level of at least one gene, reducing spots on skin, fading superficial spots on skin, fading brown spots on skin or any combination thereof includes: administering to a subject in need thereof a composition including an Ampelopsis grossedentata leaf extract. The at least one gene is at least one of a TYR gene, a TYRP1 gene, an MC1R gene and an M1TF gene.

In some embodiments, the individual or the subject may be human.

In some embodiments, effective amount of the Ampelopsis grossedentata leaf extract in the composition is 0.8 g/d.

In some embodiments, the prepared composition may be a pharmaceutical composition, an edible composition for non-medical purposes, a cosmetic composition, or a skin care product composition.

In some embodiments, when the composition is the pharmaceutical composition, the pharmaceutical composition includes an effective amount of Ampelopsis grossedentata leaf extract. The pharmaceutical composition may be made into a dosage form suitable for being administrated enterally, parenterally, orally, or topically using techniques well known to those skilled in the art.

In some embodiments, the dosage form suitable for enteral or oral administration may be, but is not limited to, tablets, troches, lozenges, pills, capsules, dispersible powder, granules, solutions, suspensions, emulsions, syrups, elixirs, slurries or the like.

In some embodiments, the dosage form suitable for parenteral or topical administration may be, but is not limited to, an injection (for example, a sterile aqueous solution or dispersion), sterile powder, an external preparation or the like.

In some embodiments, the injection may be administered by, but is not limited to, intraperitoneal injection, subcutaneous injection, intraepidermal injection, intradermal injection, intramuscular injection, intravenous injection, or intralesional injection.

In some embodiments, the pharmaceutical composition including an effective amount of Ampelopsis grossedentata leaf extract may further include pharmaceutically acceptable carriers that are widely used in pharmaceutical manufacturing technology. In some embodiments, the pharmaceutically acceptable carrier may be one or more of the following: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The type and quantity of carriers used are within the professional and routine technical scope of those skilled in the art. The solvent as the pharmaceutically acceptable carrier may be water, normal saline, phosphate buffered saline (PBS) or alcohol containing aqueous solutions.

In some embodiments, the pharmaceutical composition including an effective amount of Ampelopsis grossedentata leaf extract may be manufactured into an external preparation suitable for topical application to skin using techniques well known to those skilled in the art. In some embodiments, the external preparation includes, but is not limited to, emulsion, gel, ointment, cream, patch, liniment, powder, aerosol, spray, lotion, serum, paste, foam, drop, suspension, salve, or bandage.

In some embodiments, when the pharmaceutical composition is the external preparation, the pharmaceutical composition may be prepared by mixing an effective amount of Ampelopsis grossedentata leaf extract with a base well known to those skilled in the art.

In some embodiments, the base may include one or more of the following additives: water, alcohols, glycols, hydrocarbons, (such as petroleum, jelly, and white petrolatum), wax (such as paraffin and yellow wax), preserving agents, antioxidants, surfactants, absorption enhancers, stabilizing agents, gelling agents (such as carbopol® 974P, microcrystalline cellulose and carboxymethylcellulose), active agents, humectants, odor absorbers, fragrances, pH adjusting agents, chelating agents, emulsifiers, occlusive agents, emollients, thickeners, solubilizing agents, penetration enhancers, anti-irritants, colorants, propellants, and the like. The use and quantity of these additives are within the professional and routine technical scope of those skilled in the art.

In some embodiments, when the composition is the edible composition for non-medical purposes, the edible composition includes an effective amount of Ampelopsis grossedentata leaf extract. The edible composition may be in the form of powder, granules, solutions, colloids, or pasty fluids.

In some embodiments, the edible composition containing an Ampelopsis grossedentata leaf extract for non-medical purposes may be food products or food additives.

In some embodiments, the edible composition containing an Ampelopsis grossedentata leaf extract may be beverages, fermented foods, bakery products, health foods, or dietary supplements, etc. In some embodiments, the edible composition containing an Ampelopsis grossedentata leaf extract may further include an adjuvant. For example, the adjuvant may be Maltodextrin, malic acid, sucralose, citric acid, fruit flavor, honey flavor, steviol glycoside, or a combination thereof. The type and quantity of adjuvants used are within the professional and routine technical scope of those skilled in the art.

In some embodiments, the food additives may be seasonings, sweeteners, flavors, pH adjusting agents, emulsifiers, coloring materials or stabilizers, or the like.

In some embodiments, when the composition is the cosmetic composition or the skin care product composition, the cosmetic composition or the skin care product composition includes an effective amount of Ampelopsis grossedentata leaf extract.

In some embodiments, the cosmetic composition or the skin care product composition containing an Ampelopsis grossedentata leaf extract may be in any one of the following forms: toner, gel, gel mask, mud mask, lotion, cream, lipstick, foundation, pressed powder, setting powder, cleansing oil, makeup removing lotion, facial cleanser, body wash, shampoo, hair conditioner, sunscreen, hand cream, nail polish, perfume, essence, and facial mask.

In some embodiments, the cosmetic composition or the skin care product composition containing an Ampelopsis grossedentata leaf extract may further include externally acceptable components as required. In some embodiments, the externally acceptable components may be emulsifiers, penetration enhancers, emollients, solvents, excipients, antioxidants, or a combination thereof.

Unless otherwise specified in the following examples, the experimental steps are performed at room temperature (25-30° C.) and normal pressure (1 atm).

### Example 1: Preparation of Ampelopsis grossedentata Leaf Extract

### A. Materials:

1. Ampelopsis grossedentata tea leaves (Origin: Zhangjiajie, China). Ampelopsis grossedentata tea leaves were made from the leaves of Ampelopsis grossedentata that undergo plucking, fresh leaf airing, fixation, rolling, and withering.
2. Secondary water, also known as RO (Reverse Osmosis) water or redistilled water, hereinafter referred to as “water”.

### B. Preparation Flow:

1. Dried Ampelopsis grossedentata tea leaves were grinded to form Ampelopsis grossedentata leaf powder.
2. Water was heated to reach 85±5° C., the Ampelopsis grossedentata leaf powder was added and soaked in the water at 85±5° C. for 60 min to form primary extract containing solids. The weight ratio of the added Ampelopsis grossedentata leaf powder to the water was 1:20.
3. The cooled primary extract was filtered with a 400-mesh filter to remove the solids (namely the Ampelopsis grossedentata leaf powder subjected to extraction) to obtain filtrate.
4. The temperature of a concentrator (Model: Rotavapor R-100; Brand: BUCHI) was set to be 60±5° C., the filtrate was concentrated at reduced pressure with the concentrator. The concentration was stopped when the Degrees Brix of the filtrate reached 6.5±0.5° Bx, so as to obtain the Ampelopsis grossedentata leaf extract.

### Example 2: Ultraviolet Ray Resistance and Defense Test I

### A. Materials and Instruments:

1. Cell line: Human skin fibroblasts, purchased from BCRC (Bioresource Collection and Research Center), cell No.: 60153, hereafter referred to as CCD-966Sk cells.
2. Cell culture medium: MEM (Minimum essential medium) (purchased from Gibco, Product No.: 11095080), added with 10% Fetal Bovine Serum (purchased from Gibco, Product No.: 10437-028), 1% Penicillin-streptomycin (purchased from Gibco, Product No.: 15140122), 1 mM sodium pyruvate (purchased from Gibco, Product No.: 11360-070), 1.5 g/L sodium bicarbonate (purchased from Sigma, Product No.: S5761-500G) and 0.1 mM non-essential amino acids (purchased from Gibco, Product No.: 11140050).
3. 4 mg/mL MTT reagent: Prepared with MTT (3-(4,5-Dimethylthiazol-2-yl)-2,5-diphenyltetrazolium bromide, purchased from AMERSCO, Product No.: 0793-5G) and DPBS.
4. DMSO (Dimethyl sulfoxide): purchased from ECHO, Product No.: DA1101-000000-72EC.
5. UV Crosslinker: purchased from Cleaver Scientific, model: CL-508.
6. ELISA reader: purchased from BioTek Company (USA).

### B. Test Flow:

1. The CCD-966Sk cells were inoculated into a 96-well culture plate containing a cell culture medium at a density of 5×10³ cells per well, and were cultured at 37° C. for 24 h. The CCD-966Sk cells were divided into three test groups, namely a blank group, a control group and an experimental group. Test was repeatedly performed for each group in triplicate.
2. After culturing for 24 h, the cell culture medium of each group was replaced, the control group and the experimental group were transferred into the UV Crosslinker, and the CCD-966Sk cells in the control group and the experimental group were irradiated with UVA (ultraviolet ray with irradiation energy of 12 J/cm²) for 1 h.
3. After irradiating, the cell culture medium in each group was replaced with an experiment culture medium, each group was cultured at 37° C. for 24 h. The experiment culture medium in the blank group and the control group were the cell culture medium without a sample, and the experiment culture medium in the experimental group was the cell culture medium containing 0.03125% (v/v) of Ampelopsis grossedentata leaf extract prepared in Example 1.
4. After culturing for 24 h, 15 μL of 4 mg/mL MTT reagent was added into each group, and each group was reacted at 37° C. for 4 h.
5. After reacting for 4 h, the reacted experiment culture medium of each group was removed, 50 μL of DMSO was added into each group to dissolve Formazan crystals. Each group was placed on an orbital shaker for shaking and reacting for 10 min.
6. The absorbance of 570 nm (OD₅₇₀ value) of each group was measured with the ELISA reader.

### C. Test Results:

The relative cell viability of all groups was calculated according to the following formula: relative cell viability (%)=(OD₅₇₀ value of each group/OD₅₇₀ value of the blank group)×100%. Since the experiment was conducted in triplicate, the test results were presented as an average of the results of the triplicate experiments.

The statistically significant differences between the test results of the blank group and other groups, as well as between the test results of the control group and other groups, were obtained by statistical analysis using student t-test. In the figure, “*” means that the p value is less than 0.05 as compared to the blank group, “**” means that the p value is less than 0.01 as compared to the blank group, and “***” means that the p value is less than 0.001 as compared to the blank group; and “#” means that the p value is less than 0.05 as compared to the control group, “##” means that the p value is less than 0.01 as compared to the control group, and “## #” means that the p value is less than 0.001 as compared to the control group.

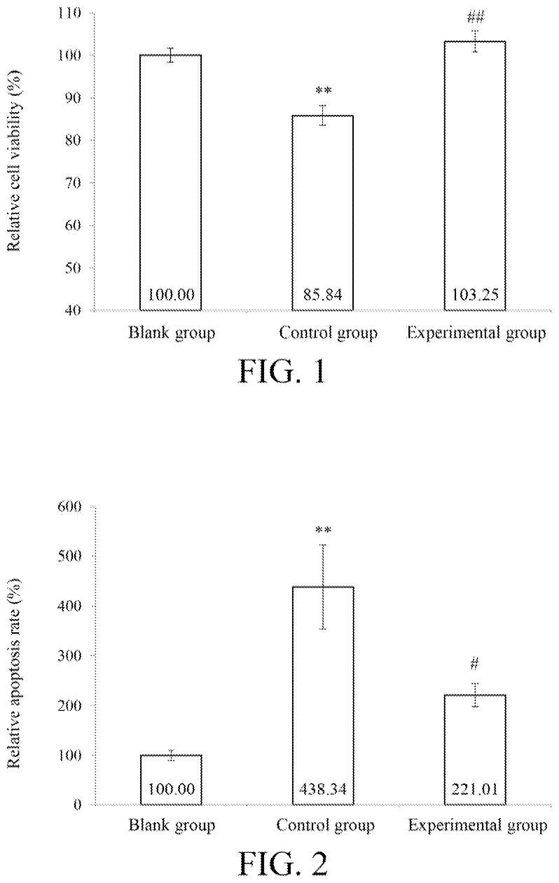

As shown in FIG. 1, cells in the blank group were not stimulated with UVA and were also not treated with the sample, so the test result of the blank group represented the expression of the cells under a normal physiological metabolism condition. Under a condition that the relative cell viability of the blank group was set to be 100%, the relative cell viability of the control group was 85.84%, and the relative cell viability of the experimental group was 103.25%. That is, compared with the blank group, the relative cell viability of the control group was significantly reduced by about 14.16% after the cells in the control group were stimulated with UVA. Compared with the control group, the relative cell viability of the experimental group was significantly improved by about 20.3% after the cells in the experimental group were stimulated with UVA and treated with the Ampelopsis grossedentata leaf extract. Compared with the blank group, the relative cell viability of the experimental group was improved by about 3.3% after the cells in the experimental group were stimulated with UVA and treated with the Ampelopsis grossedentata leaf extract.

Therefore, the Ampelopsis grossedentata leaf extract can significantly improve the reduced cell viability of the skin fibroblasts after UVA stimulation, and further improve the cell viability of the skin fibroblasts to a level higher than that of the skin fibroblasts under the normal physiological metabolism condition. The UVA has strong penetrating power, the UVA can penetrate through the skin surface layer and damage the dermis layer structure to damage skin cells, and cause skin laxity, depression, wrinkles or melanogenesis. That is, experiments have shown that the Ampelopsis grossedentata leaf extract has the effects of resisting and defending the ultraviolet ray, reducing the damage to skin caused by the ultraviolet ray, improving the viability of the skin cells under the ultraviolet ray, and providing the ultraviolet ray resistance capability for the skin. The Ampelopsis grossedentata leaf extract has the effects of resisting the damage to cells caused by the ultraviolet ray, defending the cell injury and improving the cell viability.

### Example 3: Ultraviolet Ray Resistance and Defense Test II

### A. Materials and Instruments:

1. Cell line: human skin fibroblasts, purchased from BCRC, cell No.: 60153, hereafter referred to as CCD-966Sk cells.
2. Cell culture medium: MEM (purchased from Gibco, Product No.: 11095080), added with 10% Fetal Bovine Serum (purchased from Gibco, Product No.: 10437-028), 1% Penicillin-streptomycin (purchased from Gibco, Product No.: 15140122), 1 mM sodium pyruvate (purchased from Gibco, Product No.: 11360-070), 1.5 g/L sodium bicarbonate (purchased from Sigma, Product No.: S5761-500G) and 0.1 mM non-essential amino acids (purchased from Gibco, Product No.: 11140050).
3. Trypsin: prepared by diluting 10× trypsin (purchased from Gibco, Product No.: 15400-054) and 9 times its volume of DPBS.
4. Dead Cell Apoptosis Kit with Annexin V Alexa Fluor™ 488 & Propidium Iodide (purchased from Invitrogen, Product No.: V13241): including Propidium iodide (PI) and Annexin V.
5. UV Crosslinker: purchased from Cleaver Scientific, model: CL-508.
6. Flow cytometer: purchased from BD.

### B. Test Flow:

1. The CCD-966Sk cells were inoculated into a 6-well culture plate containing 2 mL cell culture medium in each well at a density of 2×10⁵ cells per well, and were cultured at 37° C. for 24 h. The CCD-966Sk cells were divided into three test groups, namely a blank group, a control group and an experimental group. Test was repeatedly performed for each group in triplicate.
2. After culturing for 24 h, the cell culture medium in each group was replaced with an experiment culture medium. The experiment culture medium in the blank group and the control group were the cell culture medium without a sample, and the experiment culture medium in the experimental group was the cell culture medium containing 0.03125% (v/v) of Ampelopsis grossedentata leaf extract prepared in Example 1. Then, each group was cultured at 37° C. for 24 h.
3. After culturing for 24 h, the control group and the experimental group were transferred into the UV Crosslinker, and the CCD-966Sk cells in the control group and the experimental group were irradiated with UVA (ultraviolet ray with irradiation energy of 8 J/cm²) for 1 h.
4. Propidium iodide (dilution ratio: 1:250) and Annexin V (dilution ratio: 1:250) were added into each group, and each group was subjected to reaction at room temperature for 15 min. The test flow after the reaction was performed in the dark.
5. After reaction, the experiment culture medium of each group was removed, and each group was rinsed with PBS twice.
6. After rinsing, trypsin was added into each well for a reaction for 5 min. After reaction, the cell culture medium was added to stop the reaction. Then suspension cells and the cell culture medium in each well were collected into a corresponding centrifuge tube, and each centrifuge tube was centrifuged to precipitate the cells.
7. Supernatant in each centrifuge tube was removed, the precipitated cells were washed with PBS once, and PBS was added into each centrifuge tube to resuspend the cells to form cell suspension.
8. The parameter of excitation light of the flow cytometer was set to be 488 nm, and the parameter of emission light of the flow cytometer was set to be 530 nm and 575 nm. A fluorescence signal of each group was detected with the flow cytometer.

### C. Test Results:

The relative apoptosis rate of all groups was calculated according to the following formula: relative apoptosis rate (%)=(fluorescence signal of each group/fluorescence signal of the blank group)×100%. Since the experiment was conducted in triplicate, the test results were presented as an average of the results of the triplicate experiments.

The statistically significant differences between the test results of the blank group and other groups, as well as between the test results of the control group and other groups, were obtained by statistical analysis using student t-test. In the figure, “*” means that the p value is less than 0.05 as compared to the blank group, “**” means that the p value is less than 0.01 as compared to the blank group, and “***” means that the p value is less than 0.001 as compared to the blank group; and “#” means that the p value is less than 0.05 as compared to the control group, “##” means that the p value is less than 0.01 as compared to the control group, and “## #” means that the p value is less than 0.001 as compared to the control group.

As shown in FIG. 2, cells in the blank group were not stimulated with UVA and were also not treated with the sample, so the test result of the blank group represented the expression of the cells under a normal physiological metabolism condition. Under a condition that the relative apoptosis rate of the blank group was set to be 100%, the relative apoptosis rate of the control group was 438.34%, and the relative apoptosis rate of the experimental group was 221.01%. That is, compared with the blank group, the relative apoptosis rate of the control group was significantly improved by about 338.34% after the cells in the control group were stimulated with UVA. Compared with the control group, the relative apoptosis rate of the experimental group was significantly reduced by about 49.6% after the cells in the experimental group were stimulated with UVA and treated with the Ampelopsis grossedentata leaf extract.

Therefore, the Ampelopsis grossedentata leaf extract can significantly reduce the increased apoptosis rate of the skin fibroblasts after UVA stimulation. Through the combination of Annexin V and propidium iodide, cells in the early stage and the late stage of cell apoptosis can be observed at the same time, and the phenomenon and the degree of cell apoptosis can be observed. That is, experiments have shown that the Ampelopsis grossedentata leaf extract has the effects of reducing the damage to skin caused by ultraviolet ray and reducing the mortality of the skin cells under ultraviolet ray. The Ampelopsis grossedentata leaf extract has the effects of resisting the damage to cell caused by the ultraviolet ray, protecting the cells, reducing the cell apoptosis and providing the ultraviolet ray resistance capability for the skin.

### Example 4: MMP9 (Matrix Metalloproteinase 9) Level Test

A. Materials and instruments:
1. Cell line: human skin fibroblasts, purchased from BCRC, cell No.: 60153, hereafter referred to as CCD-966Sk cells.
2. Cell culture medium: MEM (purchased from Gibco, Product No.: 11095080), added with 10% fetal bovine serum (purchased from Gibco, Product No.: 10437-028), 1 mM sodium pyruvate (purchased from Gibco, Product No.: 11360070), 1.5 g/L sodium bicarbonate (purchased from Sigma, Product No.: S5761-500G), and 0.1 mM non-essential amino acids (purchased from Gibco, Product No.: 11140050).
3. Trypsin: prepared by diluting 10× trypsin (purchased from Gibco, Product No.: 15400-054) and 9 times its volume of DPBS.
4. MMP9 kit (ELISA Kit for Matrix Metalloproteinase 9): purchased from USCN, Product No.: SEA553Hu.
5. RIPA reagent: purchased from Invitrogen, Product No.: 89900.
6. ELISA reader: purchased from BioTek Company (USA).
7. Ultraviolet ray irradiation chamber: purchased from Vilber.

### B. Test Flow:

1. The CCD-966Sk cells were inoculated into a 6-well culture plate containing 2 mL cell culture medium in each well at a density of 2×10⁵ cells per well, and were cultured at 37° C. for 24 h. The CCD-966Sk cells were divided into three test groups, namely a blank group, a control group and an experimental group. Test was repeatedly performed for each group in triplicate.
2. After culturing for 24 h, the cell culture medium in each group was replaced with an experiment culture medium. The experiment culture medium in the blank group and the control group were the cell culture medium without a sample, and the experiment culture medium in the experimental group was the cell culture medium containing 0.03125% (v/v) of Ampelopsis grossedentata leaf extract prepared in Example 1.
3. The control group and the experimental group were transferred into the ultraviolet ray irradiation chamber, and the CCD-966Sk cells in the control group and the experimental group were irradiated with UVB (ultraviolet ray with irradiation energy of 50 J/cm²) for 40 min.
4. After irradiation, each group was cultured at 37° C. for 48 h.
5. After culturing, the experiment culture medium of each group was removed, and each group was rinsed with PBS once.
6. After rinsing, trypsin was added into each well for a reaction for 3 min. After reaction, the cell culture medium was added to stop the reaction. Then suspension cells and the cell culture medium in each well were collected into a corresponding centrifuge tube, and each centrifuge tube was centrifuged to precipitate the cells.
7. Supernatant in each centrifuge tube was removed, the precipitated cells were washed with PBS once, then 50 μL of RIPA reagent was added into each centrifuge tube to resuspend the cells. Later, each centrifuge tube was centrifuged.
8. Supernatant in each centrifuge tube was collected, and the supernatant was subjected to measure the MMP9 level in the CCD-966Sk cells of each group with the MMP9 kit. After the supernatant of each group was treated according to a test flow provided by the MMP9 kit, the absorbance of 450 nm (OD₄₅₀ value) of each well was measured with the ELISA reader.

### C. Test Results:

The relative MMP9 level of all groups was calculated according to the following formula: relative MMP9 level (%)=(OD₄₅₀ value of each group/OD₄₅₀ value of the blank group)×100%. Since the experiment was conducted in triplicate, the test results were presented as an average of the results of the triplicate experiments.

The statistically significant differences between the test results of the blank group and other groups, as well as between the test results of the control group and other groups, were obtained by statistical analysis using student t-test. In the figure, “#” means that the p value is less than 0.05 as compared to the blank group, “##” means that the p value is less than 0.01 as compared to the blank group, and “## #” means that the p value is less than 0.001 as compared to the blank group, and “*” means that the p value is less than 0.05 as compared to the control group, “**” means that the p value is less than 0.01 as compared to the control group, and “***” means that the p value is less than 0.001 as compared to the control group.

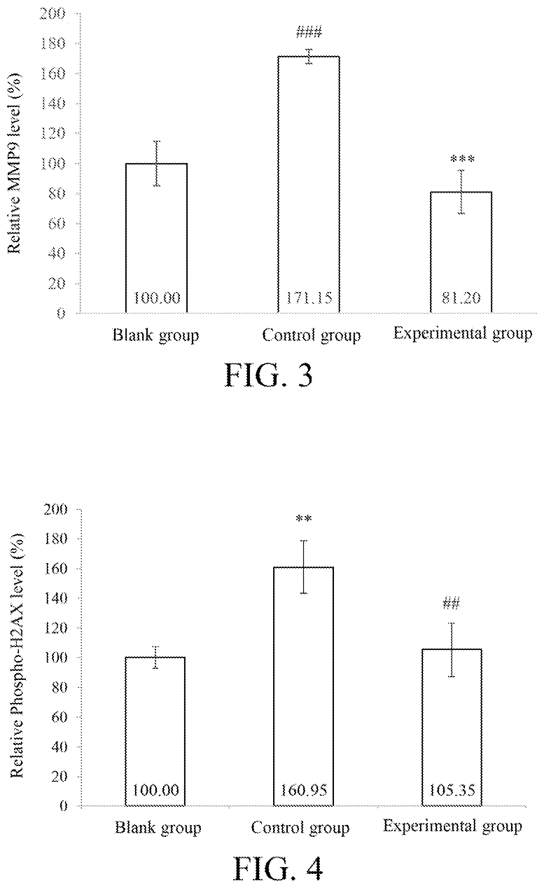

As shown in FIG. 3, cells in the blank group were not stimulated with UVB and were also not treated with the sample, so the test result of the blank group represented the expression of the cells under a normal physiological metabolism condition. Under a condition that the relative MMP9 level in the blank group was set to be 100%, the relative MMP9 level in the control group was 171.15%, and the relative MMP9 level in the experimental group was 81.20%. That is, compared with the blank group, the relative MMP9 level in the control group was significantly improved by about 71.15% after the cells of the control group were stimulated with UVB. Compared with the blank group, the relative MMP9 level in the experimental group was reduced by about 18.80% after the cells in the experimental group were stimulated with UVB and treated with the Ampelopsis grossedentata leaf extract. Compared with the control group, the relative MMP9 level in the experimental group was significantly reduced by about 52.6% after the cells in the experimental group were stimulated with UVB and treated with the Ampelopsis grossedentata leaf extract.

Therefore, the Ampelopsis grossedentata leaf extract can significantly reduce the increased MMP9 level in the skin fibroblasts after UVB stimulation, and the MMP9 level was further reduced to a level far lower than the MMP9 level in the skin fibroblasts under the normal physiological metabolism condition. After long-term exposure to ultraviolet rays, the UVB can cause increased expression level of the MMP9. MMP is a protease, which can decompose collagen. A large amount of MMP easily causes skin tissue arrangement loosen, skin laxity and skin wrinkles. That is, experiments have shown that the Ampelopsis grossedentata leaf extract has the effects of inhibiting and/or reducing the generation of skin cell MMP9 protein caused by ultraviolet ray, repairing the damage to skin cells caused by ultraviolet ray, stabilizing the skin structure, and inhibiting skin laxity and skin wrinkles caused by ultraviolet ray. The Ampelopsis grossedentata leaf extract has the effects of reducing the level of the cell MMP9 and repairing damaged cells. In addition, the decomposition of the MMP may also cause tumors, and the MMP also participates in angiogenesis and may cause the growth and diffusion of cancer cells.

### Example 5: DNA Damage Test

### A. Materials and Instruments:

1. Cell line: human skin fibroblasts, purchased from BCRC, cell No.: 60153, hereafter referred to as CCD-966Sk cells.
2. Cell culture medium: MEM (purchased from Gibco, Product No.: 11095080), added with 10% fetal bovine serum (purchased from Gibco, Product No.: 10437-028), 1% antibiotics (purchased from Invivogen, Product No.: ant-mpp) and 1 mM sodium pyruvate (purchased from Gibco, Product No. 11360-070).
3. H₂O₂ solution: prepared with H₂O₂ (purchased from Sigma, Product No.: 1.08600) and DPBS.
4. Trypsin: prepared by diluting 10× trypsin (purchased from Gibco, Product No.: 15400-054) and 9 times its volume of DPBS.
5. RIPA reagent: purchased from Invitrogen, Product No.: 89900.
6. Human Phospho-H₂AX (S139) ELISA Kit: purchased from RayBio, Product No.: PEL-H₂AX-S139.
7. ELISA reader: purchased from BioTek Company (USA).

### B. Test Flow:

1. The CCD-966Sk cells were inoculated into a 6-well culture plate containing a cell culture medium at a density of 2×10⁵ cells per well, and were cultured at 37° C. for 24 h. The CCD-966Sk cells were divided into three test groups, namely a blank group, a control group and an experimental group. Test was repeatedly performed for each group in triplicate.
2. After culturing for 24 h, the cell culture medium in each group was replaced with an experiment culture medium. The experiment culture medium in the blank group and the control group were the cell culture medium without a sample, and the experiment culture medium in the experimental group was the cell culture medium containing 0.0625% (v/v) of Ampelopsis grossedentata leaf extract prepared in Example 1. Then, each group was reacted at 37° C. for 1 h.
3. After reacting for 1 h, H₂O₂ solution was added into the control group and the experimental group to reach the final concentration of the H₂O₂ solution of 1 mM, and each group was reacted at 37° C. for 1 h.
4. After reacting, the experiment culture medium of each group was removed, and each group was rinsed with PBS once.
5. After rinsing, trypsin was added into each well for a reaction for 3 min. After reaction, the cell culture medium was added to stop the reaction. Then suspension cells and the cell culture medium in each well were collected into a corresponding centrifuge tube, and each centrifuge tube was centrifuged to precipitate the cells.
6. Supernatant in each centrifuge tube was removed, the precipitated cells were washed with PBS once, then 150 μL of RIPA reagent was added into each centrifuge tube to resuspend the cells. Later, each centrifuge tube was centrifuged.
7. Supernatant in each centrifuge tube was collected, and the supernatant was subjected to determine the Phospho-H₂AX level in the CCD-966Sk cells in each group with the human Phospho-histone H₂AX (site S139) ELISA kit. After the supernatant of each group was treated according to the test flow provided by the human Phospho-histone H₂AX (site S139) ELISA kit, the absorbance of 450 nm (OD₄₅₀ value) of each well was measured with the ELISA reader.
   C. Test results:

The relative Phospho-H₂AX level in all groups was calculated according to the following formula: relative Phospho-H₂AX level (%)=(OD₄₅₀ value of each group/OD₄₅₀ value of the blank group)×100%. Since the experiment was conducted in triplicate, the test results were presented as an average of the results of the triplicate experiments.

The statistically significant differences between the test results of the blank group and other groups, as well as between the test results of the control group and other groups, were obtained by statistical analysis using student t-test. In the figure, “*” means that the p value is less than 0.05 as compared to the blank group, “**” means that the p value is less than 0.01 as compared to the blank group, and “***” means that the p value is less than 0.001 as compared to the blank group; and “#” means that the p value is less than 0.05 as compared to the control group, “##” means that the p value is less than 0.01 as compared to the control group, and “## #” means that the p value is less than 0.001 as compared to the control group.

As shown in FIG. 4, cells in the blank group were not treated with a sample and were also not stimulated with H₂O₂, so the test result of the blank group represented the expression of the cells under a normal physiological metabolism condition. Under a condition that the relative Phospho-H₂AX level in the blank group was set to be 100%, the relative Phospho-H₂AX level in the control group was 160.95%, and the relative Phospho-H₂AX level in the experimental group was 105.35%. That is, compared with the blank group, the relative Phospho-H₂AX level in the control group was significantly improved by about 60.95% after the cells of the control group were stimulated with H₂O₂. Compared with the control group, the relative Phospho-H₂AX level of the experimental group was significantly reduced by about 34.5% after the cells in the experimental group were stimulated with H₂O₂ and treated with the Ampelopsis grossedentata leaf extract.

Therefore, the Ampelopsis grossedentata leaf extract can significantly reduce the increased Phospho-H₂AX level in the skin fibroblasts after H₂O₂ stimulation. H₂O₂ can induce aerobic metabolism of cells, so that ROS is generated. ROS in the cells can attack macromolecules such as nucleic acid to cause DNA damage. When double-strand DNA is broken, Phospho-H₂AX will be generated. That is, experiments have shown that the Ampelopsis grossedentata leaf extract has the effects of reducing damage to skin cell DNA caused by oxidation and preventing and/or reducing damage or breakage of the skin cell DNA. The Ampelopsis grossedentata leaf extract has the effects of reducing DNA damage or breakage, repairing DNA, repairing damage caused by oxidation pressure and promoting physiological metabolism. The Ampelopsis grossedentata leaf extract has the effects of improving the antioxidant capability of the skin and enhancing the skin resistance.

### Example 6: Elastin Test

### A. Materials and Instruments:

1. Cell line: human skin fibroblasts, purchased from ATCC (American Type Culture Collection), cell No.: CRL-1881, hereafter referred to as CCD-966Sk cells.
2. Cell culture medium: MEM (purchased from Gibco, Product No.: 11095080), added with 10% fetal bovine serum (purchased from Gibco, Product No.: 10437-028), 1% Penicillin-streptomycin (purchased from Gibco, Product No. 15140122) and 1 mM sodium pyruvate (purchased from Gibco, Product No. 11360-070).
3. Trypsin: prepared by diluting 10× trypsin (purchased from Gibco, Product No.: 15400-054) and 9 times its volume of DPBS.
4. Fastin™ Elastin Assay kit: purchased from Biocolor, Product No.: F2000.
5. ELISA reader: purchased from BioTek Company (USA).

### B. Test Flow:

1. The CCD-966Sk cells were inoculated into a 6-well culture plate containing 2 mL cell culture medium in each well at a density of 1×10⁵ cells per well, and were cultured at 37° C. for 24 h. The CCD-966Sk cells were divided into two test groups, namely a blank group and an experimental group. Test was repeatedly performed for each group in triplicate.
2. After culturing for 24 h, the cell culture medium in each group was replaced with an experiment culture medium. The experiment culture medium in the blank group was the cell culture medium without a sample, and the experiment culture medium in the experimental group was the cell culture medium containing 0.0625% (v/v) of Ampelopsis grossedentata leaf extract prepared in Example 1. Then, each group was cultured at 37° C. for 48 h.
3. After culturing for 48 h, the experimental culture medium of each group was removed, and each group was rinsed with PBS once.
4. After rinsing, trypsin was added into each well for a reaction for 3 min. After reaction, the cell culture medium was added to stop the reaction. Then suspension cells and the cell culture medium in each well were collected into a corresponding centrifuge tube, and each centrifuge tube was centrifuged to precipitate the cells.
5. Supernatant in each centrifuge tube was removed, the precipitated cells were washed with PBS once, and 300 μL of PBS was added into each centrifuge tube to resuspend the cells to form cell suspension.
6. The elastin level in the CCD-966Sk cells in each group was determined with the Fastin™ Elastin Assay kit. After the cell suspension in each group was treated according to a test flow provided by the Fastin™ Elastin Assay kit, the absorbance of 513 nm (OD₅₁₃ value) of each well was measured with the ELISA reader.

### C. Test Results:

The relative elastin level of all groups was calculated according to the following formula: relative elastin level (%)=(OD₅₁₃ value of each group/OD₅₁₃ value of the blank group)×100%. Since the experiment was conducted in triplicate, the test results were presented as an average of the results of the triplicate experiments.

The statistically significant difference between the test results of the blank group and the experimental group was obtained by statistical analysis using student t-test. In the figure, “*” means that the p value is less than 0.05 as compared to the blank group, “**” means that the p value is less than 0.01 as compared to the blank group, and “***” means that the p value is less than 0.001 as compared to the blank group.

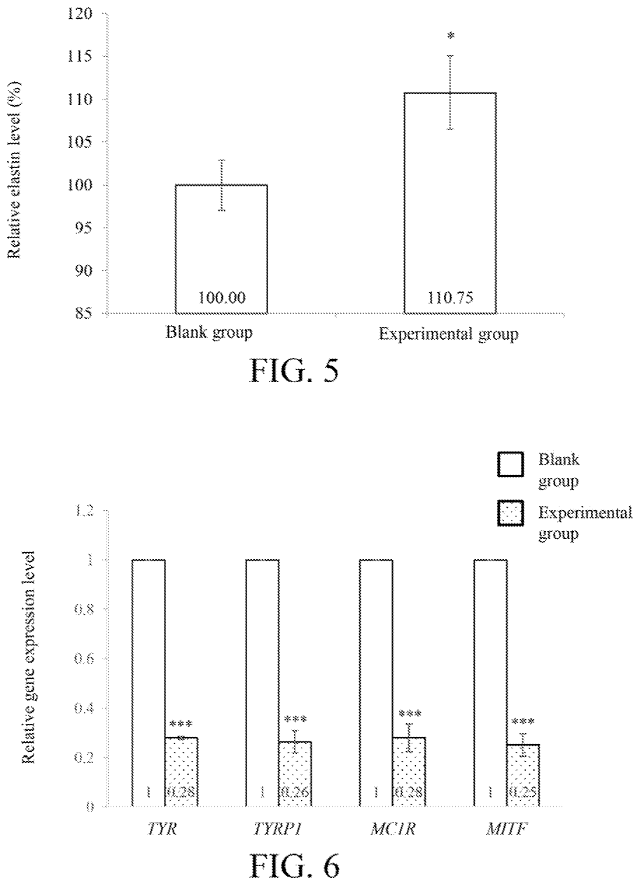

As shown in FIG. 5, cells in the blank group were not treated with a sample, so the test result of the blank group represented the expression of the cells under a normal physiological metabolism condition. Under a condition that the relative elastin level of the blank group was set to be 100%, the relative elastin level of the experimental group was 110.75%. That is, compared with the blank group, the relative elastin level of the experimental group was significantly increased by about 10.75% after the cells of the experimental group were treated with the Ampelopsis grossedentata leaf extract.

Therefore, the Ampelopsis grossedentata leaf extract can significantly increase the elastin level in the skin fibroblasts. The elastin plays a role in consolidating collagen and elastic fiber structures in the skin so as to maintain the elasticity of the skin. That is, experiments have shown that the Ampelopsis grossedentata leaf extract has the effects of increasing the skin elastin expression, stabilizing the skin scaffold and improving the skin elasticity. The Ampelopsis grossedentata leaf extract has the effect of increasing the elastin level.

### Example 7: Genetic Test

### A. Materials and Instruments:

1. Cell line: human melanoma cells, purchased from ATCC, cell No.: CRL-1872, hereinafter referred to as A375.S2 cells.
2. Cell culture medium: MEM-Non-Essential Amino Acids (purchased from Gibco, Product No.: 11095080), added with 10% fetal bovine serum (purchased from Gibco, Product No.: 10437-028) and 1 mM sodium pyruvate (purchased from Gibco, Product No.: 11360-070).
3. RNA extraction kit: purchased from TAN Bead, Product No.: 301538.
4. SuperScript® III reverse transcriptase: purchased from Invitrogen Company, Product No.: 18080-051.
5. ABI StepOnePlus™ Real-Time PCR system: purchased from Thermo Fisher Scientific.
6. KAPA SYBR FAST qPCR (2×) Kit: purchased from KAPA Biosystems, Product No.: KM4102.

### B. Test Flow:

1. The A375.S2 cells were inoculated into a 6-well culture plate containing 2 mL of cell culture medium in each well at a density of 1.5×10⁵ cells per well, and were cultured at 37° C. for 24 h. The A375.S2 cells were divided into two test groups, namely a blank group and an experimental group. Test was repeatedly performed for each group in triplicate.
2. After culturing for 24 h, the cell culture medium in each group was replaced with an experiment culture medium. The experiment culture medium in the blank group was the cell culture medium without a sample, and the experiment culture medium in the experimental group was the cell culture medium containing 0.0625% (v/v) of Ampelopsis grossedentata leaf extract prepared in Example 1. Then, each group was cultured at 37° C. for 24 h.
3. After culturing for 24 h, the A375.S2 cells in each group were collected. Then, RNA in the A375.S2 cells of each group was extracted with the RNA extraction kit.
4. 1,000 ng of the extracted RNA from each group was used as a template, and the extracted RNA was reversely transcribed into corresponding cDNA with the SuperScript® III reverse transcriptase.
5. A quantitative real-time reverse transcription polymerase chain reaction was carried out on the cDNA in each group with the ABI StepOnePlus™ Real-Time PCR system using KAPA SYBR FAST qPCR (2×) Kit and primers pairs in Table 1 respectively to observe the expression level and melting curve of various target genes of A375.S2 cells in the blank group and the experimental group. The setting conditions of instrument for quantitative real-time reverse transcription polymerase chain reaction were that: reaction at 95° C. for 20 s, reaction at 95° C. for 3 s, reaction at 60° C. for 30 s, 40 circles in total.
6. The relative expression level of the target gene was measured by a 2^{-ΔΔCt} method. The relative expression level was defined as the fold change of the RNA expression level of the target gene in the experimental group or the blank group relative to the RNA expression level of the same gene in the blank group. The mRNA expression level of the gene could be indirectly quantified by performing the quantitative real-time reverse transcription polymerase chain reaction on the cDNA, and the expression level of protein encoded by the gene was further deduced. The 2^{-ΔΔCt} method was used for calculating the fold change according to the following formula by taking a circulating threshold of a GAPDH (glyceraldehyde 3-phosphate dehydrogenase) gene as a circulating threshold (Ct) of a reference gene for internal control:

$$Δ Ct = {CT}_{Target gene of experimental group / Target gene of blank group} - {Ct}_{GAPDH}$$

$$ΔΔ Ct = Δ {Ct}_{Target gene of experimental group} - Δ {Ct}_{Target gene of blank group}$$

$$Fold change = {2}^{- Δ Δ Ct average}$$

TABLE 1

| Target | Primer | Sequence | |
|---|---|---|---|
| gene | name | number | Sequence |
| TYR | TYR-F | SEQ ID NO: 1 | CTCAAAGCAGCATGCACAAT |
| | TYR-R | SEQ ID NO: 2 | GCCCAGATCTTTGGATGAAA |
| TYRP1 | TYRP1-F | SEQ ID NO: 3 | GACACGCCTCCTTTTTATTCCA |
| | TYRP1-R | SEQ ID NO: 4 | ATGGGTTTGTCCCCCTGTTC |
| MC1R | MC1R-F | SEQ ID NO: 5 | CATCATCGACCCCCTCATCTAC |
| | MC1R-R | SEQ ID NO: 6 | CAGGAACCAGACCACACAATATCA |
| MITF | MITF-F | SEQ ID NO: 7 | GCCTCCAAGCCTCCGATAAG |
| | MITF-R | SEQ ID NO: 8 | GCACTCTCTGTTGCATGAACT |

### C. Test Results:

The relative target gene expression level of all groups was calculated according to the following formula: relative target gene expression level=(target gene expression level of each group/target gene expression level of the blank group). Since the experiment was conducted in triplicate, the test results were presented as an average of the results of the triplicate experiments.

The statistically significant difference between the test results of the blank group and the experimental group was obtained by statistical analysis using student t-test. In the figure, “*” means that the p value is less than 0.05 as compared to the blank group, “**” means that the p value is less than 0.01 as compared to the blank group, and “***” means that the p value is less than 0.001 as compared to the blank group.

As shown in FIG. 6, cells in the blank group were not treated with a sample, so the test result of the blank group represented the expression of the cells under a normal physiological metabolism condition. Under a condition that the relative expression level of TYR, TYRP1, MC1R and M1TF genes in the blank group were set to be 1 fold, the relative expression level of the TYR gene in the experimental group was 0.28 fold; the relative expression level of the TYRP1 gene in the experimental group was 0.26 fold; the relative expression level of the MC1R gene in the experimental group was 0.28 fold; and the relative expression level of the M1TF gene in the experimental group was 0.25 fold. That is, compared with the blank group, the relative expression level of the TYR gene in the experimental group was significantly reduced by about 72% after the cells in the experimental group were treated with the Ampelopsis grossedentata leaf extract. Compared with the blank group, the relative expression level of the TYRP1 gene in the experimental group was significantly reduced by about 74% after the cells in the experimental group were treated with the Ampelopsis grossedentata leaf extract. Compared with the blank group, the relative expression level of the MC1R gene in the experimental group was significantly reduced by about 72% after the cells in the experimental group were treated with the Ampelopsis grossedentata leaf extract. Compared with the blank group, the relative expression level of the M1TF gene in the experimental group was significantly reduced by about 75% after the cells in the experimental group were treated with the Ampelopsis grossedentata leaf extract.

Therefore, the Ampelopsis grossedentata leaf extract can significantly reduce the expression level of TYR, TYRP1, MC1R and M1TF genes of melanoma cells. The TYR gene is responsible for producing tyrosinase, and the TYRP1 gene is responsible for producing tyrosinase-related protein 1. Both the tyrosinase and the protein participate in the generation of melanin. The MC1R gene is responsible for producing a receptor, namely a melanocortin 1 receptor, which plays an important role in normal pigmentation and stimulates the melanocytes to generate eumelanin. The M1TF gene is responsible for producing protein, namely a melanocyte inducing transcription factor, which controls the generation of melanin. That is, experiments have shown that the Ampelopsis grossedentata leaf extract has the effects of inhibiting and/or reducing the level of the tyrosinase, the tyrosinase-related protein 1, the melanocortin 1 receptor and the melanocyte inducing transcription factor of the melanoma cells. The Ampelopsis grossedentata leaf extract has the effects of inhibiting and/or reducing melanin production in the melanoma cells. The Ampelopsis grossedentata leaf extract has the effects of inhibiting and/or reducing melanogenesis, whitening and brightening skin, making the skin pure, bright and white, and improving the skin brightness.

### Example 8: Anti-Inflammation Test

### A. Materials and Instruments:

1. Cell line: mouse macrophages, purchased from ATCC, cell No.: TIB-71, hereinafter referred to as RAW 264.7 cells.
2. Cell culture medium: DMEM (Dulbecco's Modified Eagle Medium) (purchased from Gibco, Product No.: 12100-046), added with 10% fetal bovine serum (purchased from Gibco, Product No.: 10437-028), 1% antibiotics (purchased from Thermo, Product No.: 15240062) and 4 mM L-glutamine (purchased from Gibco, Product No.: 25030081).
3. Serum-free culture medium: DMEM (purchased from Gibco, Product No.: 12100-046), added with 1% antibiotics (purchased from Thermo, Product No.: 15240062) and 4 mM L-glutamine (purchased from Gibco, Product No.: 25030081).
4. LPS (Lipopolysaccharide): purchased from Sigma, Product No.: SI-L2880-25 MG.
5. Griess reagent: prepared with a reagent A and a reagent B in the Griess reagent kit (purchased from Life technologies, Product No.: 1445263) with a volume ratio of 1:1.
6. ELISA reader: purchased from BioTek Company (USA).

### B. Test Flow:

1. The RAW 264.7 cells were inoculated into a 96-well culture plate A containing 200 μL of cell culture medium in each well at a density of 1×10⁴ cells per well, and were cultured at 37° C. for 24 h. The A375.S2 cells were divided into three test groups, namely a blank group, a control group and an experimental group. Test was repeatedly performed for each group in quadruplicate.
2. After culturing for 24 h, the cell culture medium in each group was replaced with an experiment culture medium. The experiment culture medium in the blank group was the cell culture medium without a sample and LPS; the experiment culture medium in the control group was the serum-free culture medium containing 200 ng/ml LPS; and the experiment culture medium in the experimental group was the serum-free culture medium containing 200 ng/mL LPS and 0.0625% (v/v) of Ampelopsis grossedentata leaf extract prepared in Example 1. Then, each group was reacted at 37° C. for 24 h.
3. 130 μL of redistilled water was added into each well in another 96-well culture plate B.
4. After reacting for 24 h, 150 μL of reacted experiment culture medium from each well of each group was took out and added into the corresponding well of the 96-well culture plate B containing 130 μL of redistilled water.
5. 20 μL of Griess reagent was added into each well of the 96-well culture plate B, and each group was reacted for 30 min in the dark.
6. The absorbance of 548 nm (OD₅₄₈ value) of each well was measured with the ELISA reader. The higher the OD₅₄₈ value, the higher the level of nitric oxide.

### C. Test Results:

The relative nitric oxide level of all groups was calculated according to the following formula: relative nitric oxide level (%)=(OD₅₄₈ value of each group/OD₅₄₈ value of the control group)×100%. Since the experiment was carried out four times, the test results were presented as an average of the results of the quadruplicate experiments.

The statistically significant differences between the test results of the blank group and other groups, as well as between the test results of the control group and other groups, were obtained by statistical analysis using student t-test. In the figure, “#” means that the p value is less than 0.05 as compared to the blank group, “##” means that the p value is less than 0.01 as compared to the blank group, and “## #” means that the p value is less than 0.001 as compared to the blank group; and “*” means that the p value is less than 0.05 as compared to the control group, “**” means that the p value is less than 0.01 as compared to the control group, and means that the p value is less than 0.001 as compared to the control group.

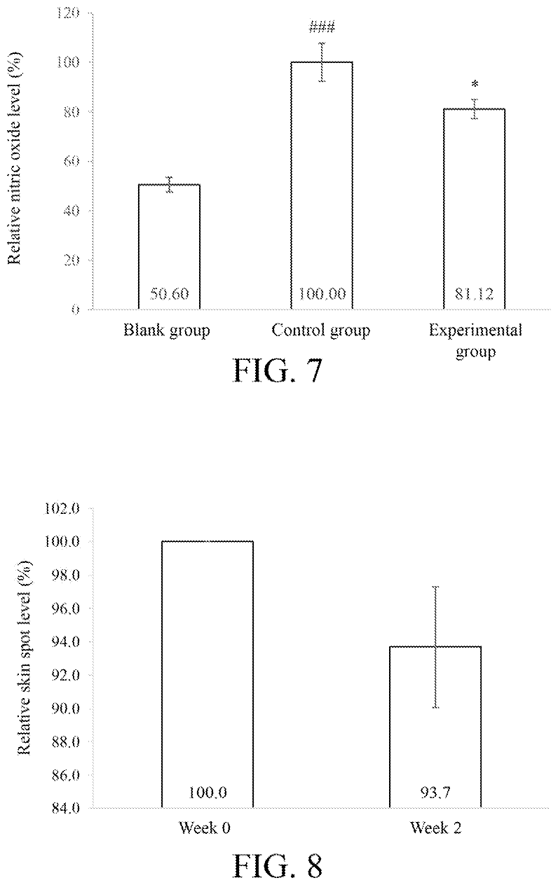

As shown in FIG. 7, cells in the control group were stimulated with LPS and were not treated with a sample, so the test result of the control group represented the expression of the cells under an inflammation condition. Under a condition that the relative nitric oxide level in the control group was set to be 100%, the relative nitric oxide level in the blank group was 50.60%, and the relative nitric oxide level in the experimental group was 81.12%. That is, compared with the blank group, the relative nitric oxide level in the control group was significantly increased by about 97.63% after the cells in the control group were stimulated with LPS. Compared with the control group, the relative nitric oxide level in the experimental group was significantly reduced by about 20.3% after the cells in the experimental group were stimulated with the LPS and treated with the Ampelopsis grossedentata leaf extract.

Therefore, the Ampelopsis grossedentata leaf extract can significantly reduce the increased nitric oxide level in macrophage after LPS stimulation. When the macrophage is in contact with a pathogen, the generation of the nitric oxide is promoted. The LPS is used for simulating the pathogen to promote the macrophage to generate the nitric oxide, and then an inflammatory reaction is caused. That is, experiments have shown that the Ampelopsis grossedentata leaf extract has the effects of reducing the nitric oxide level and reducing the inflammatory reaction. The Ampelopsis grossedentata leaf extract has an anti-inflammation effect and can provide an anti-inflammation capability for skin.

### Example 9: Human Subject Test

### A. Test Process:

9 healthy adult subjects over 20 years old were given one bottle of test drink every day for 2 consecutive weeks (i.e., 14 days). The test drink contained 0.8 g of the Ampelopsis grossedentata leaf extract prepared in Example 1 and 49.2 g of water. In addition, the subjects underwent skin detection before consumption (hereinafter referred to as week 0) and after 14 days of consumption (hereinafter referred to as week 2).

Skin detection was based on different skin detection items, using corresponding instruments and measurement methods to record the value of facial skin and take photos before and after consumption. Skin detecting items included skin spots, skin brown spots, skin wrinkles, skin texture and skin hydration. Moreover, no matter at week 0 or week 2, when detecting, the temperature and humidity of the test area where the subjects was located were consistent to reduce the impact of external factors such as temperature and humidity on the skin.

Skin spots were detected on the facial skin of a same subject before and after consumption using a VISIA Complexion Analysis System purchased from Canfield Scientific Company in the United States. This System took high-resolution skin images through visible light (white light), and used software to analyze the number and area of pigment spots visible to the naked eye to obtain a value that represented the spot status of the skin (hereinafter referred to as the skin spot level value). The higher the obtained skin spot level value, the more serious the skin spot level. Then, the relative skin spot level was calculated using the following formula: relative skin spot level (%)=(skin spot level value of each group/skin spot level value before consumption)×100%.

Skin brown spots were detected on the facial skin of a same subject before and after consumption using a VISIA Complexion Analysis System purchased from Canfield Scientific Company in the United States. This System used RBX polarized light technology to take pictures of facial skin and detect melanin spots in the dermis that were invisible to the naked eye to obtain a value that represented the brown spot status of the skin (hereinafter referred to as the skin brown spot level value). The higher the obtained skin brown spot level value, the more serious the skin brown spot level. Then, the relative skin brown spot level was calculated using the following formula: relative skin brown spot level (%)=(skin brown spot level value of each group/skin brown spot level value before consumption)×100%.

Skin wrinkles were detected on the facial skin of a same subject before and after consumption using a VISIA Complexion Analysis System purchased from Canfield Scientific Company in the United States. This System took pictures of facial skin through a high-resolution camera lens. By illuminating visible light (white light) and detecting changes in skin shadows, this system can analyze and calculate the length and depth of wrinkles to obtain a value that represented the wrinkle status of the skin (hereinafter referred to as the skin wrinkle level value). The higher the obtained skin wrinkle level value, the more serious the skin wrinkle level. Then, the relative skin wrinkle level was calculated using the following formula: relative skin wrinkle level (%)=(skin wrinkle level value of each group/skin wrinkle level value before consumption)×100%.

Skin texture was detected on the facial skin of a same subject before and after consumption using a VISIA Complexion Analysis System purchased from Canfield Scientific Company in the United States. This System took pictures of facial skin through a high-resolution camera lens. By illuminating standard white light, this system can detect the depressions and ridges of the skin for analysis and calculation to obtain a value that represented the skin roughness (hereinafter referred to as the skin texture level value). The higher the obtained skin texture level value, the more serious the skin roughness. Then, the relative skin texture level was calculated using the following formula: relative skin texture level (%)=(skin texture level value of each group/skin texture level value before consumption)×100%.

Skin hydration was detected on the facial skin of a same subject before and after consumption using a C+K Multi Probe Adapter System Corneometer® CM825 (Skin hydration detection probe) purchased from Courage+Khazaka electronic Company in Germany. This detection probe measured based on the principle of capacitance. When the hydration changed, the capacitance value of the skin also changed, so a value that represented the skin hydration (hereinafter referred to as the skin hydration value) can be obtained by determining the skin capacitance value. The higher the obtained skin hydration value, the higher the skin hydration. Then, the relative skin hydration was calculated using the following formula: relative skin hydration (%)=(skin hydration value of each group/skin hydration value before consumption)×100%.

### B. Test Results:

The statistically significant differences between the test results at week 0 and week 2 were obtained by statistical analysis using student t-test. In the figure, “*” means that the p value is less than 0.05 as compared to week 0, “**” means that the p value is less than 0.01 as compared to week 0, and “***” means that the p value is less than 0.001 as compared to week 0.

As shown in FIG. 8, the skin spot level values of 9 subjects measured before consumption were regarded as 100% of relative skin spot level. Then, the relative skin spot level at week 2 (namely after consumption of the test drink for 2 consecutive weeks) was 93.7%. That is, compared with week 0, the relative skin spot level of the subjects can be reduced by 6.3% after consumption of the Ampelopsis grossedentata leaf extract for 2 consecutive weeks. The percentage of subjects with improvement reached 77.8% (7 subjects). Therefore, the Ampelopsis grossedentata leaf extract can really reduce skin spots. That is, experiments have shown that the Ampelopsis grossedentata leaf extract has the effects of fading visible spots and superficial spots on the skin.

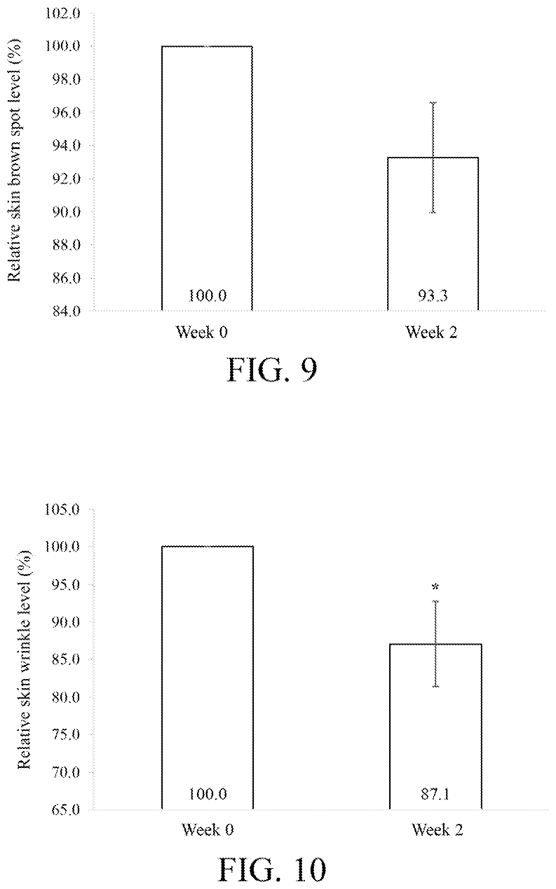

As shown in FIG. 9, the skin brown spot level values of 9 subjects measured before consumption were regarded as 100% of relative skin brown spot level. Then, the relative skin brown spot level at week 2 (namely after consumption of the test drink for 2 consecutive weeks) was 93.3%. That is, compared with week 0, the relative skin brown spot level of the subjects can be reduced by 6.7% after consumption of the Ampelopsis grossedentata leaf extract for 2 consecutive weeks. The percentage of subjects with improvement reached 77.8% (7 subjects). Therefore, the Ampelopsis grossedentata leaf extract can really reduce skin brown spots. That is, experiments have shown that the Ampelopsis grossedentata leaf extract has the effects of reducing and/or fading deep spots on the skin and helping the skin to clean the spots from the deep layer.

As shown in FIG. 10, the skin wrinkle level values of 9 subjects measured before consumption were regarded as 100% of relative skin wrinkle level. Then, the relative skin wrinkle level at week 2 (namely after consumption of the test drink for 2 consecutive weeks) was 87.1%. That is, compared with week 0, the relative skin wrinkle level of the subjects can be significantly reduced by 12.9% after consumption of the Ampelopsis grossedentata leaf extract for 2 consecutive weeks. The percentage of subjects with improvement reached 88.9% (8 subjects). Therefore, the Ampelopsis grossedentata leaf extract can really reduce skin wrinkles. That is, experiments have shown that the Ampelopsis grossedentata leaf extract has the effects of reducing skin wrinkles, smoothing skin fine lines and smoothing skin textures.

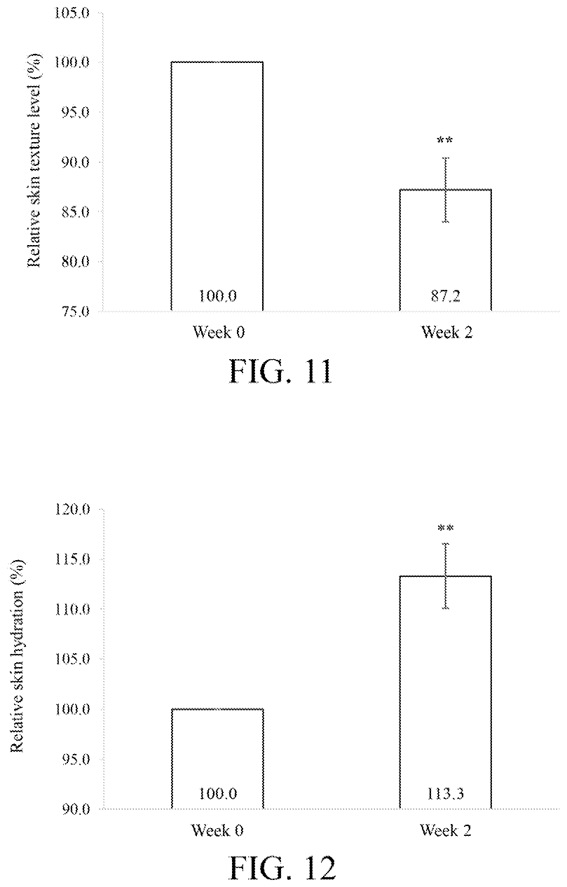

As shown in FIG. 11, the skin texture level values of 9 subjects measured before consumption were regarded as 100% of relative skin texture level. Then, the relative skin texture level at week 2 (namely after consumption of the test drink for 2 consecutive weeks) was 87.2%. That is, compared with week 0, the relative skin texture level of the subjects can be significantly reduced by 12.8% after consumption of the Ampelopsis grossedentata leaf extract for 2 consecutive weeks. The percentage of subjects with improvement reached 100% (9 subjects). Therefore, the Ampelopsis grossedentata leaf extract can really improve skin texture. That is, experiments have shown that the Ampelopsis grossedentata leaf extract has the effects of smoothing skin texture, reducing and/or improving skin roughness, making skin finer, tender, smooth and flat, and improving skin smoothness.

As shown in FIG. 12, the skin hydration values of 9 subjects measured before consumption were regarded as 100% of relative skin hydration. Then, the relative skin hydration at week 2 (namely after consumption of the test drink for 2 consecutive weeks) was 113.3%. That is, compared with week 0, the relative skin hydration of the subjects can be significantly increased by 13.3% after consumption of the Ampelopsis grossedentata leaf extract for 2 consecutive weeks. The percentage of subjects with improvement reached 88.9% (8 subjects). Therefore, the Ampelopsis grossedentata leaf extract can really increase skin hydration. That is, experiments have shown that the Ampelopsis grossedentata leaf extract has the effect of improving skin moisture.

In conclusion, the Ampelopsis grossedentata leaf extract according to the embodiments of the present disclosure has a skin condition improving effect. In some embodiments, a use of the Ampelopsis grossedentata leaf extract according to the embodiments of the present disclosure in improving skin condition relates to a use of the Ampelopsis grossedentata leaf extract in preparation of a composition for improving skin condition, thereby providing a composition capable of realizing the skin condition improving effect on an individual when being administered to the individual. In some embodiments, a method for improving skin condition includes: administering to a subject in need thereof a composition including the Ampelopsis grossedentata leaf extract according to the embodiments of the present disclosure. That is, the composition has the skin condition improving function. That is, the composition can improve the skin condition of the individual after being administered to the individual. In some embodiments, the Ampelopsis grossedentata leaf extract or the prepared composition thereof also has one or more of the following functions: enhancing skin resistance, improving skin smoothness, and improving skin brightness. In some embodiments, methods for enhancing skin resistance, improving skin smoothness, and improving skin brightness include: administering to a subject in need thereof a composition including the Ampelopsis grossedentata leaf extract.

What is claimed is:

1. A method for enhancing skin resistance, comprising: administering to a subject in need thereof a composition comprising Ampelopsis grossedentata leaf extract, wherein the Ampelopsis grossedentata leaf extract is obtained by extracting leaves of Ampelopsis grossedentata with water at 85±5° C. for 50-70 min.

2. The method according to claim 1, wherein the Ampelopsis grossedentata leaf extract improves an antioxidant capability of skin of the subject.

3. The method according to claim 2, wherein the Ampelopsis grossedentata leaf extract reduces DNA damage in skin cells of the subject.

4. The method according to claim 1, wherein the Ampelopsis grossedentata leaf extract provides an anti-inflammation capability for skin of the subject.

5. The method according to claim 4, wherein the Ampelopsis grossedentata leaf extract reduces nitric oxide level in skin cells of the subject.

6. The method according to claim 1, wherein the Ampelopsis grossedentata leaf extract provides an ultraviolet ray resistance capability for skin of the subject.

7. The method according to claim 6, wherein the Ampelopsis grossedentata leaf extract reduces damage to skin of the subject caused by ultraviolet ray.

8. The method according to claim 7, wherein the Ampelopsis grossedentata leaf extract improves viability of skin cells of the subject under ultraviolet ray.

9. The method according to claim 7, wherein the Ampelopsis grossedentata leaf extract reduces mortality of skin cells of the subject under ultraviolet ray.

10. The method according to claim 6, wherein the Ampelopsis grossedentata leaf extract inhibits skin laxity of the subject caused by ultraviolet ray.

11. The method according to claim 10, wherein the Ampelopsis grossedentata leaf extract reduces a level of matrix metalloproteinase 9 (MMP9) generated under ultraviolet ray in skin cells of the subject.

12. A method for improving skin smoothness, comprising: administering to a subject in need thereof a composition comprising Ampelopsis grossedentata leaf extract, wherein the Ampelopsis grossedentata leaf extract is obtained by extracting leaves of Ampelopsis grossedentata with water at 85±5° C. for 50-70 min.

13. The method according to claim 12, wherein the Ampelopsis grossedentata leaf extract reduces skin roughness of the subject.

14. The method according to claim 12, wherein the Ampelopsis grossedentata leaf extract increases elastin expression of skin cells of the subject.

15. The method according to claim 12, wherein the Ampelopsis grossedentata leaf extract reduces skin wrinkles of the subject.

16. The method according to claim 13, wherein the Ampelopsis grossedentata leaf extract improves skin texture of the subject.

17. The method according to claim 12, wherein the Ampelopsis grossedentata leaf extract increases skin hydration of the subject.

18. A method for improving skin brightness, comprising: administering to a subject in need thereof a composition comprising Ampelopsis grossedentata leaf extract, wherein the Ampelopsis grossedentata leaf extract is obtained by extracting leaves of Ampelopsis grossedentata with water at 85±5° C. for 50-70 min.

19. The method according to claim 18, wherein the Ampelopsis grossedentata leaf extract reduces melanin production in skin cells of the subject.

20. The method according to claim 19, wherein the Ampelopsis grossedentata leaf extract reduces expression level of at least one gene of the subject, and the at least one gene is at least one of a TYR gene, a TYRP1 gene, an MC1R gene, and an M1TF gene.

21. The method according to claim 18, wherein the Ampelopsis grossedentata leaf extract reduces spots on skin of the subject.

22. The method according to claim 21, wherein the Ampelopsis grossedentata leaf extract fades superficial spots on skin of the subject.

23. The method according to claim 21, wherein the Ampelopsis grossedentata leaf extract fades brown spots on skin of the subject.


SEQUENCE LISTING (8 sequences)

SEQ ID NO: 1 - DNA, 20 bp, synthetic construct
  source 1..20: mol_type = other DNA
ctcaaagcagcatgcacaat

SEQ ID NO: 2 - DNA, 20 bp, synthetic construct
  source 1..20: mol_type = other DNA
gcccagatctttggatgaaa

SEQ ID NO: 3 - DNA, 22 bp, synthetic construct
  source 1..22: mol_type = other DNA
gacacgcctcctttttattcca

SEQ ID NO: 4 - DNA, 20 bp, synthetic construct
  source 1..20: mol_type = other DNA
atgggtttgtccccctgttc

SEQ ID NO: 5 - DNA, 22 bp, synthetic construct
  source 1..22: mol_type = other DNA
catcatcgaccccctcatctac

SEQ ID NO: 6 - DNA, 24 bp, synthetic construct
  source 1..24: mol_type = other DNA
caggaaccagaccacacaatatca

SEQ ID NO: 7 - DNA, 20 bp, synthetic construct
  source 1..20: mol_type = other DNA
gcctccaagcctccgataag

SEQ ID NO: 8 - DNA, 21 bp, synthetic construct
  source 1..21: mol_type = other DNA
gcactctctgttgcatgaact